(19) United States
(12) Patent Application Publication
(10) Pub. No.: US 2024/0327374 A1
(43) Pub. Date: Oct. 3, 2024

(54) LENALIDOMIDE PROCESS

(71) Applicants: Akshay Kant Chaturvedi, Bhiwadi (IN); Mohammad Baqer, Gugugram (IN); Bijan Kumar Panda, Bhubanesware (IN); Satyaveer, Bareilly (IN); Deepali Chaturvedi, Bhiwadi (IN); Saroj Bala, Gurugram (IN); Yogesh Tripathi, Varanasi (IN)

(72) Inventors: Akshay Kant Chaturvedi, Bhiwadi (IN); Mohammad Baqer, Gugugram (IN); Bijan Kumar Panda, Bhubanesware (IN); Satyaveer, Bareilly (IN); Deepali Chaturvedi, Bhiwadi (IN); Saroj Bala, Gurugram (IN); Yogesh Tripathi, Varanasi (IN)

(21) Appl. No.: 18/129,074

(22) Filed: Mar. 31, 2023

(51) Int. Cl. C07D 401/04

(52) U.S. Cl. CPC C07D 401/04; C07B 2200/13

(57) ABSTRACT

The present invention provides to an improved process for the preparation of new and high purity crystalline Form SRA of 3-(4-amino-1-oxoisoindolin-2-yl) piperidine-2, 6-dione of formula (I)

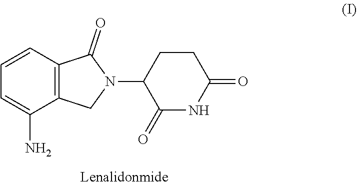 [SMILES: Nc1cccc2c1CN(C1CCC(=O)NC1=O)C2=O]

The pure crystalline Form SRA of compound of formula (I) obtained by the process of present invention is having purity of greater than 99.9% (area % by HPLC).

Lenalidomide (I) pure crystalline Form SRA is useful in the treatment of cancer, inflammatory diseases, autoimmune diseases and Multiple myeloma.

### FIELD OF THE INVENTION

The present invention relates to an improved process for the preparation of pure crystalline Form SRA of Lenalidomide (I).

### BACKGROUND OF THE INVENTION

Lenalidomide (I) is chemically named as 3-(7-amino-3-oxo-1H-isoindol-2-yl) piperidine-2, 6-dione.

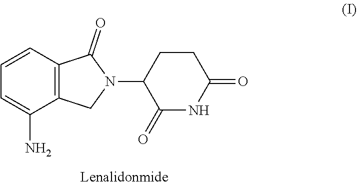 [SMILES: Nc1cccc2c1CN(C1CCC(=O)NC1=O)C2=O]

Lenalidomide is a cancer drug and is also known by its brand name, “REVLIMID”. It is used in the treatment for multiple myeloma and blood disorders called myelodysplastic syndromes (MDS).

Lenalidomide appears to works by enhancing the immune system that indirectly attacks the cancer cells. It prevents the growth of new blood vessels within the tumor. It also restricts the production of chemical messengers (cytokine and growth factor) which are responsible for the growth of cancer cells.

Muller, et al U.S. Pat. No. 5,635,517B1 first discloses Lenalidomide and its preparation process, which follows the pathway as given in the Scheme-1.

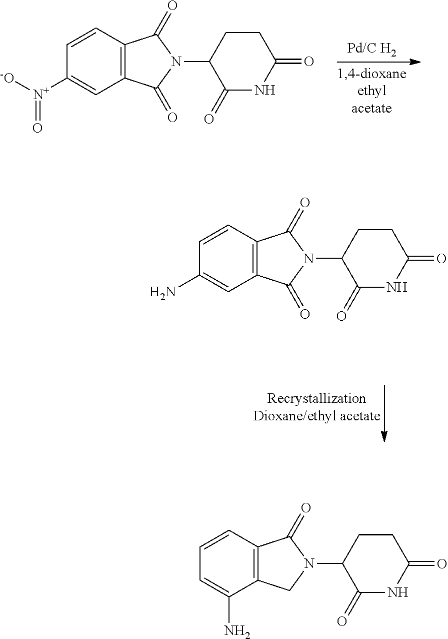

### Scheme-1: Process as Per U.S. Pat. No. 5,635,517B1

Muller, et al in U.S. Pat. No. 5,635,517B1 described Lenalidomide process comprising of reducing 1,3-dioxo-2-(2,6-dioxopiperidin-3-yl)-4-nitroisoindoline (1 g, 3.3 mmol) using 10% Pd/C in 1,4-dioxane (200 ml) was hydrogenated at 50 psi for 6.5 hours followed by obtained residue was crystallized from ethyl acetate (20 mL) to give 0.62 g (69%) of 1,3-dioxo-2-(2,6-dioxopiperidin-3-yl)-4-aminoisoindoline as an orange solid and recrystallization from dioxane/ethyl acetate gave yellow solid of Lenalidomide.

Muller, et al in WO2005023192A2 discloses polymorphic Forms of Lenalidomide as Form-A, Form-B, Form-C, Form-D, Form-E, Form-F, Form-G and Form-H. It was represented in this patent application that the polymorphic Form-A is anhydrous form, Polymorphic Form-B is hemihydrate crystalline solid. Polymorphic Form-C is disclosed as a hemisolvate solid and Polymorphic Form-D is solvated with water and acetonitrile. Form-E is dihydrate and Form-F can be obtained by complete dehydration of Form-E. Polymorphic Form-G is a un-solvated material and Form-H is a crystalline solid, hydrated with about 0.25 moles of water.

Surya Narayana et al in US20110021567A1 disclose the process for the preparation of pure Lenalidomide. This patent application also relates to an enriched, amorphous form of Lenalidomide which follows the preparation pathway as given in the Scheme-2:

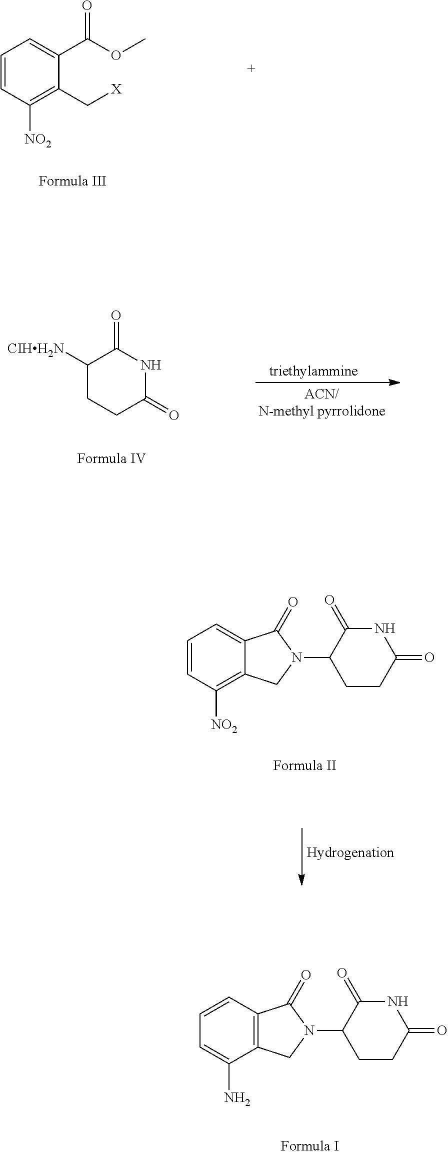

### Scheme-2: Process as Per US20110021567A1

Konakanchi et al in U.S. Pat. No. 8,877,932 B2 discloses anhydrous Lenalidomide Form-I. Further acid additional salt of Lenalidomide and their polymorphic form are disclosed in patent application WO 2011050962A1.

Zhang Heshehg in US20110060010A1 discloses Polymorph IA, IB, IC, IIA, IIIA, MB, IVA, IVB, VA, VIA, VIB, VIIA, of Lenalidomide solvates of strong acids. Strong acid may be organic or inorganic acid.

Zhang Liang et al in WO2018036557A1 discloses Lenalidomide crystalline Form-J characterized by peaks in the powder x-ray diffraction pattern of Cu—Kα radiation, and its X-ray powder diffraction pattern has 2°θ expressed in degrees at 12.0±0.2°, 13.6±0.2°, 15.3±0.2°, 18.6±0.2°, 20.0±0.2°, 21.2±0.2°, 21.5±0.2°, 22.1±0.2°, 22.6±0.2°, 23.2±0.2°, 24.1±0.2°, 24.7±0.2°, 25.4±0.2°, 26.7±0.2°, 27.5±0.2° have characteristic peaks, and 2°θ has only one diffraction peak between 13.0-14.0°.

Rampalli Sriram et al in US20170107193A1 disclose the process for the preparation of anhydrous Crystalline Form-SL of Lenalidomide.

Chen, et al in U.S. Pat. No. 8,420,672B2 discloses a new crystalline Form-I of Lenalidomide as quarter hydrate (1:0.25).

Junbo; et al in US20180282297A1 discloses a dihydrate crystalline designated as Form-DH and an anhydrous Form designated as Form-α.

Alla Venkatla Rama Rao et al in WO2019092752A2 disclose crystalline Trifluoroacetic acid salt of Lenalidomide-Forms I, II, III, IV, V and WO 2015113314 A1 discloses Crystalline Form X, XI, XII, or XIII in the Lenalidomide Polymorph.

As lenalidomide [3-(4-amino-1-oxoisoindolin-2-yl)piperidine-2,6-dione] has been used in the treatment of various cancers like multiple myeloma and non-Hodgkin's lymphoma, it has also shown efficacy in other diseases/conditions, including myelodysplastic syndromes, chronic lymphocytic leukemia, and solid tumors.

It is well known fact that polymorphism of a compound affects many of the compound's physicochemical properties, such as solubility, hygroscopcity, chemical reactivity, and stability. Many of the inconsistencies encountered in drug product performance can be attributed to polymorphism. Despite the importance of polymorphism, methods of predicting the existence of possible polymorphs of a compound and conditions under which they can be formed are unreliable, and processes for producing polymorphs often fail to generate them consistently and reliably.

Hence, there exists a need of new and stable polymorphs of Lenalidomide which may provide desirable properties (e.g., improved solubility, hygroscopicity, chemical reactivity, and/or stability) to advance the development of the compound as a useful therapeutic agent. Also, there exists need for process of producing high purity and consistently producing the same polymorphic form possessing long term stability and present invention in this application addresses the need.

The inventors of the present application have now developed an improved process for the preparation of pure active pharmaceutical ingredient—as crystalline Form SRA of Lenalidomide (I) which is a stable crystalline form and devoid of process related impurities.

### SUMMARY OF THE INVENTION

Particular aspects of the present invention relates to a process for the preparation of pure crystalline Form SRA of Lenalidomide of Formula (I).

Different aspects of the present invention are summarized herein below individually.

In one aspect of the present application, the present invention relates to pure crystalline Lenalidomide (I) Form SRA

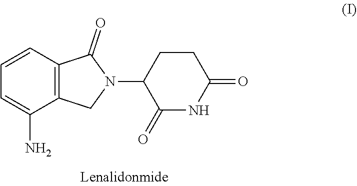 [SMILES: Nc1cccc2c1CN(C1CCC(=O)NC1=O)C2=O]

characterized by X-ray powder diffraction angle peaks selected from the XRPD peak set of 7.7, 11.2, 14.2, 15.6, 19.9, 20.3, 23.9, 25.7, 28.2, 32.3, 33.4 2θ°±0.20 2θ°.

Lenalidomide crystalline Form SRA is further characterized by DSC endothermic peak ranging between 265 to 270° C.

In further aspect, it relates to Lenalidomide crystalline Form SRA, which has an IR absorption spectrum having characteristic peaks expressed in cm⁻¹ at approximately 3410 cm⁻¹, 3347 cm⁻¹, 2962 cm⁻¹, 2912 cm⁻¹, 1642 cm⁻¹, 1607, 1452 cm⁻¹, 1411 cm⁻¹, 1209 cm⁻¹, 1163 cm⁻¹, 1014 cm⁻¹.

Crystalline Form SRA of Lenalidomide (I), of the present invention relates to the characterization of X-ray powder diffraction pattern according to FIG. 1, TGA Thermo gravimetric analysis according to FIG. 3, IR absorption spectrum according to FIG. 4, DSC isothermal pattern according to FIG. 5 and High Performance Liquid Chromatographic purity according to FIG. 6.

In yet another aspect of the present application, the present invention relates to a process for preparation of pure crystalline Form SRA of Lenalidomide (I), characterized by X-ray powder diffraction angle peaks selected from the XRPD peak set of 7.7, 11.2, 14.2, 15.6, 19.9, 20.3, 23.9, 25.7, 28.2, 32.3, 33.4 2θ°±0.20 2θ°, DSC endothermic peak ranging between 265 to 270° C. and IR absorption spectrum having characteristic peaks expressed in cm⁻¹ at approximately 3410 cm⁻¹, 3347 cm⁻¹, 2962 cm⁻¹, 2912 cm⁻¹, 1642 cm⁻¹, 1607, 1452 cm⁻¹, 1411 cm⁻¹, 1209 cm⁻¹, 1163 cm⁻¹, 1014 cm⁻¹ comprising the steps of:
i). combining the compound of formula (a) with compound of formula (b) in an organic amide solvent and triethyl amine to get 3-(4-nitro-1-Oxo-1, 3 dihydro-isoindol-2-yl)-piperidine-2, 6-dione (c)

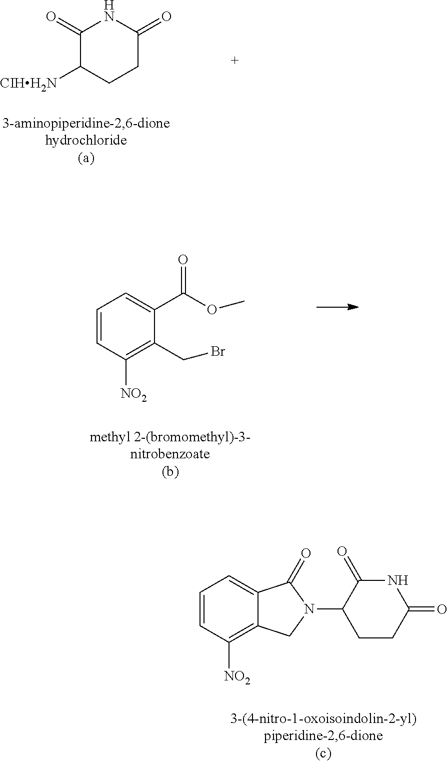

ii). reacting 3-(4-nitro-1-Oxo-1, 3 dihydro-isoindol-2-yl)-piperidine-2, 6-dione (c) with hydrogen gas at temperature ranging between 20-40° C. and normal atmospheric pressure in anhydrous medium in the presence of Palladium Carbon catalyst to get 3-(4-amino-1-oxoisoindolin-2-yl) piperidine-2,6-dione or Lenalidomide (I).

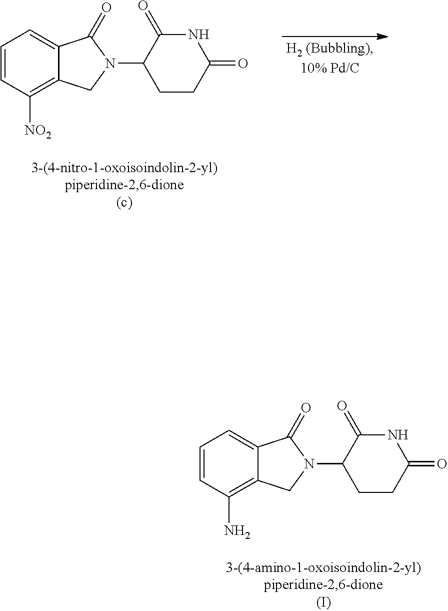 [SMILES: O=C1CCC(N2Cc3c(cccc3[N+](=O)[O-])C2=O)C(=O)N1 ; Nc1cccc2c1CN(C1CCC(=O)NC1=O)C2=O]

iii). optionally purifying compound (I) obtained in step ii) by combining with an alkylsulphonic acid in the presence of an aliphatic alcohol solvent at 25-35° C. under stirring for a time duration ranging between 30-60 minutes;
iv). filter and combine the filtrate with a mixture of an organic acid and an alkyl amine at temperature ranging between 25-35° C. for a time duration ranging between 20-50 minutes;
v). isolating the pure crystalline Form SRA.

In yet another aspect according to the present invention, it relates to highly pure crystalline Form SRA of Lenalidomide (I), having purity of greater than 99.9% (area % by HPLC).

Further, other specific aspects of the invention are detailed in the description part of the specification, wherever appropriate.

### BRIEF DESCRIPTION OF THE DRAWINGS

FIG. 1 is an illustration of X-ray powder diffraction (XRPD) pattern of the pure crystalline Form SRA of Lenalidomide.

FIG. 2 is an illustration of X-ray powder diffraction (XRPD) pattern of the pure crystalline Form SRA of Lenalidomide obtained after 6 months stability under 40° C.±2/75±5% RH condition.

FIG. 3 is an illustration of TGA thermogram of the pure crystalline Form SRA of Lenalidomide.

FIG. 4 is an illustration of IR spectrum of the pure crystalline Form SRA of Lenalidomide.

FIG. 5 is an illustration of a Differential Scanning Calorimetric (DSC) curve of the pure crystalline Form SRA of Lenalidomide.

FIG. 6 is an illustration of High performance liquid chromatography (HPLC) of the pure crystalline Form SRA of Lenalidomide.

FIG. 7 is an illustration of High performance liquid chromatography (HPLC) of the pure crystalline Form SRA of Lenalidomide obtained after 6 months stability under 40° C.±2/75±5% RH condition.

FIG. 8 is an illustration of comparative X-ray powder diffraction (XRPD) pattern of the pure crystalline Form SRA of Lenalidomide.

### DETAILED DESCRIPTION

As set forth herein, embodiments of the present invention relate to a process for preparation of highly pure crystalline Form SRA of Lenalidomide of formula (I). The present invention deals with a simple and industrially amenable process for making the compound of formula (I), which exhibits various advantages over other processes known in the state of arts. The advantages are discussed on the relevant places of further description. Individual embodiments of the present invention are detailed herein below separately.

In one embodiment according to the present application, it provides a process for preparing highly pure crystalline Form SRA of Lenalidomide of formula (I).

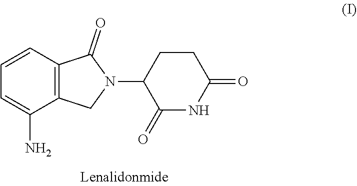 [SMILES: Nc1cccc2c1CN(C1CCC(=O)NC1=O)C2=O]

characterized by X-ray powder diffraction angle peaks selected from the XRPD peak set of 7.7, 11.2, 14.2, 15.6, 19.9, 20.3, 23.9, 25.7, 28.2, 32.3, 33.4 2θ°±0.20 2θ°.

Highly pure crystalline Form SRA of Lenalidomide of formula (I) is further characterized by DSC endothermic peak ranging between 265 to 270° C.

The characteristic peaks and their d spacing values of the new crystalline Form SRA are tabulated in the Table-1.

TABLE 1
Characteristic XRPD Peaks of Crystalline Form SRA

| Sr. No. | Angle (2θ°) ± (0.2θ) | d-Spacing Value(Å) |
|---|---|---|
| 1. | 7.7 | 11.38 |
| 2. | 11.2 | 7.91 |
| 3. | 14.2 | 6.24 |
| 4. | 15.6 | 5.67 |
| 5. | 19.9 | 4.47 |
| 6. | 20.3 | 4.38 |
| 7. | 23.9 | 3.73 |
| 8. | 25.7 | 3.47 |
| 9. | 28.2 | 3.18 |
| 10. | 32.3 | 2.76 |
| 11. | 33.4 | 2.70 |

In another embodiment, pure crystalline Form SRA of Lenalidomide, which has an IR absorption spectrum having characteristic peaks expressed in cm⁻¹ at approximately 3410 cm⁻¹, 3347 cm⁻¹, 2962 cm⁻¹, 2912 cm⁻¹, 1642 cm⁻¹, 1607, 1452 cm⁻¹, 1411 cm⁻¹, 1209 cm⁻¹, 1163 cm⁻¹, 1014 cm⁻¹.

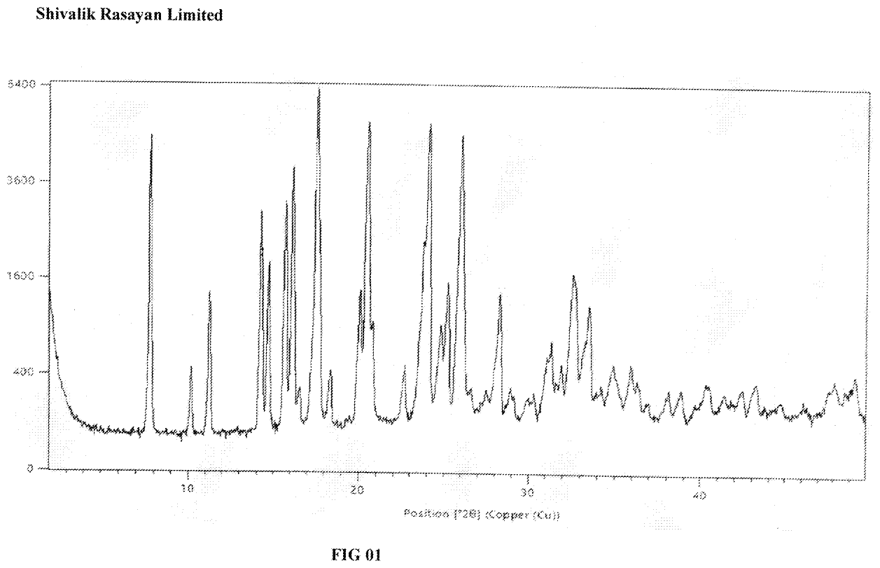

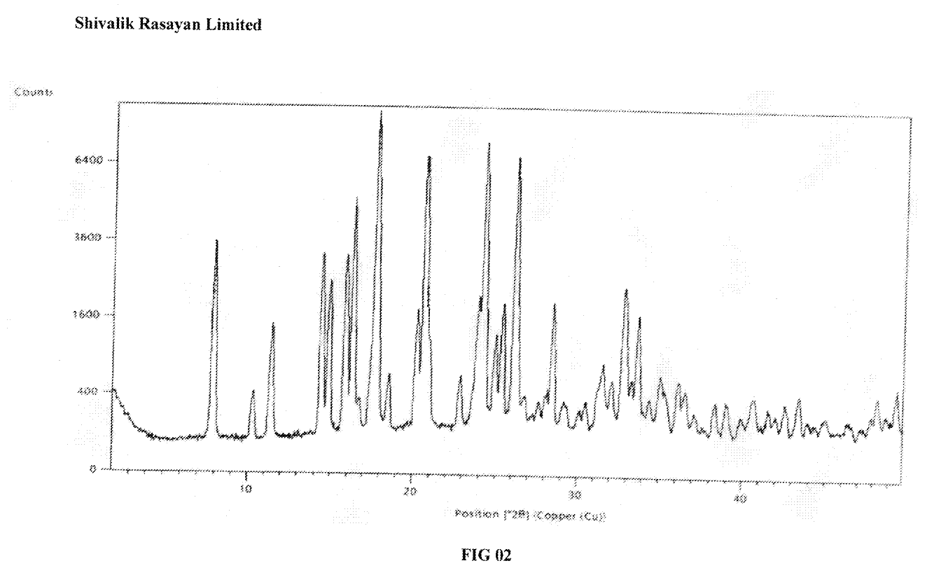

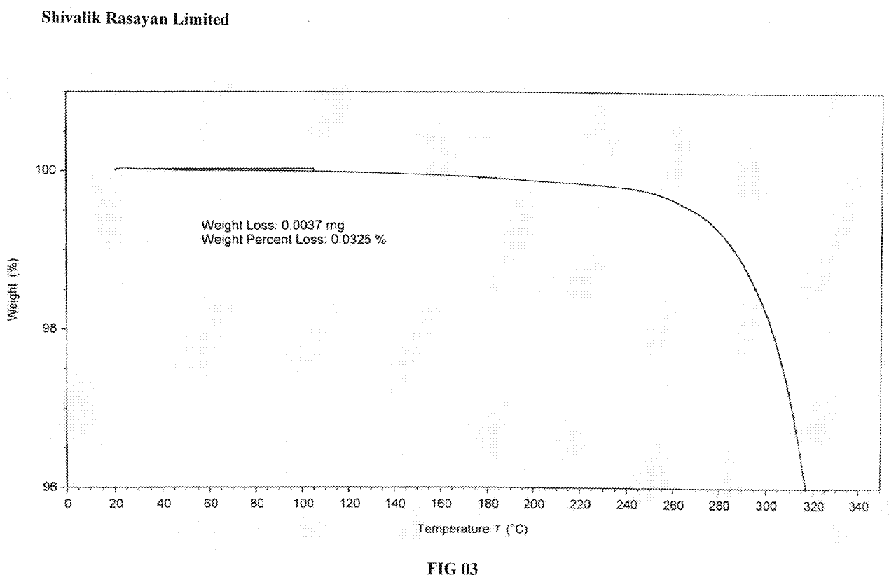

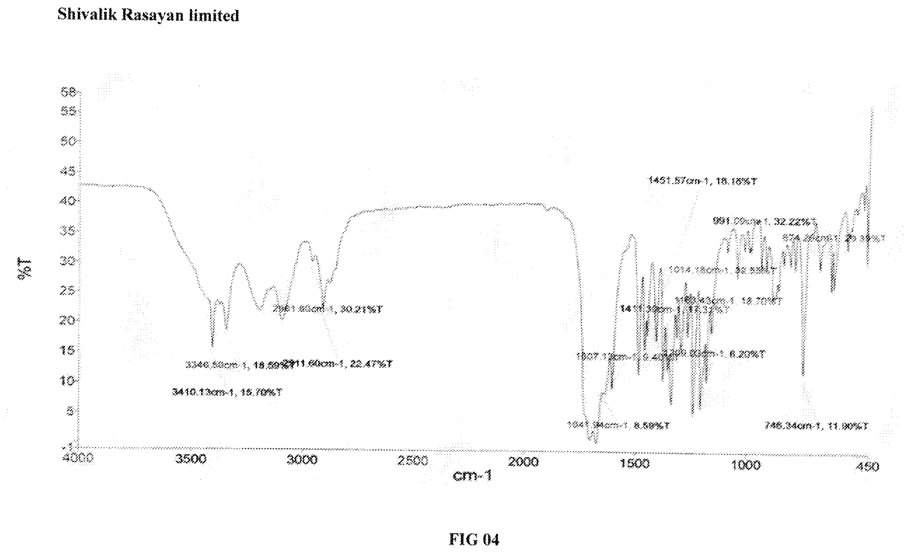

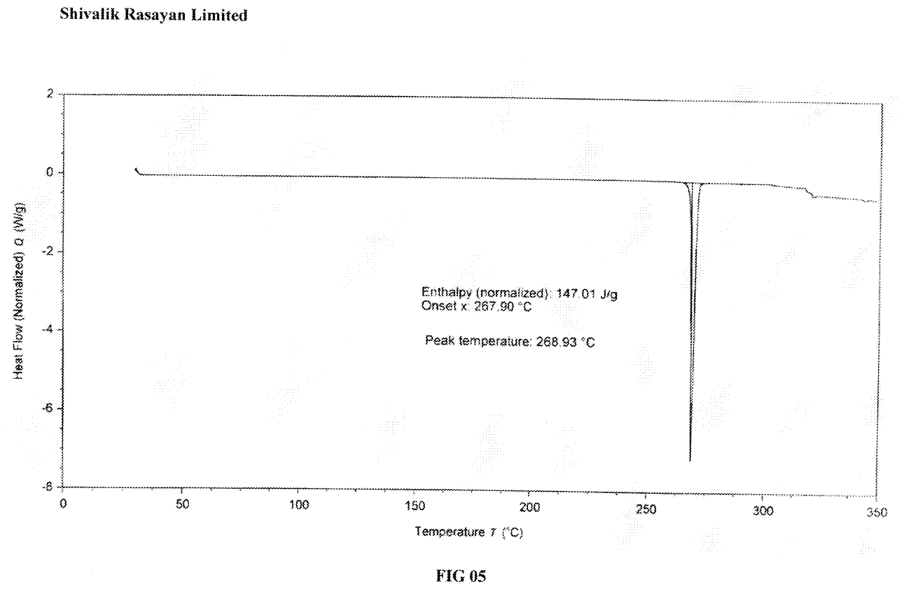

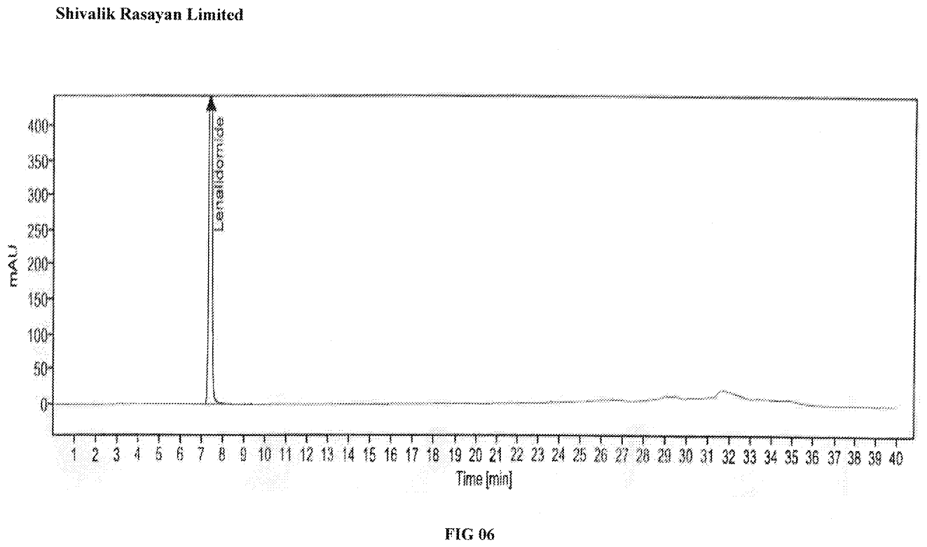

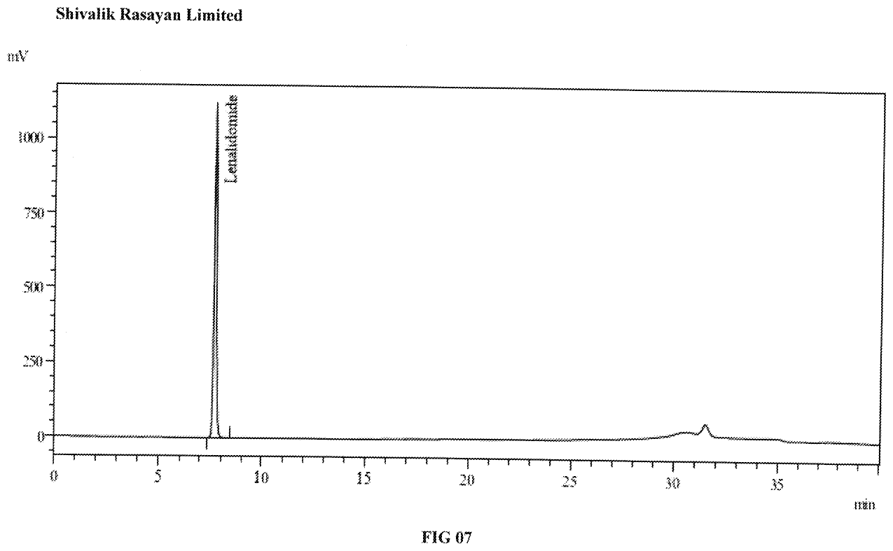

Crystalline Form SRA of Lenalidomide (I), of the present invention is further characterized by X-ray powder diffraction pattern according to FIGS. 1 and 2, TGA Thermo gravimetric analysis according to FIG. 3, IR absorption spectrum according to FIG. 4, DSC isothermal pattern according to FIG. 5 and High performance Liquid chromatographic purity according to FIGS. 6 and 7.

In yet another embodiment of the present invention, it provides a process for preparing Crystalline Form SRA of Lenalidomide is characterized by X-ray powder diffraction angle peaks selected from the XRPD peak set of 7.7, 11.2, 14.2, 15.6, 19.9, 20.3, 23.9, 25.7, 28.2, 32.3, 33.4 2θ°±0.20 20°, DSC endothermic peak ranging between 265 to 270° C. and IR absorption spectrum having characteristic peaks expressed in cm⁻¹ at approximately 3410 cm⁻¹, 3347 cm⁻¹, 2962 cm⁻¹, 2912 cm⁻¹, 1642 cm⁻¹, 1607, 1452 cm⁻¹, 1411 cm⁻¹, 1209 cm⁻¹, 1163 cm⁻¹, 1014 cm⁻¹ comprising the steps of:
i). combining the compound of formula (a) with compound of formula (b) in an organic amide solvent and triethyl amine to get 3-(4-nitro-1-Oxo-1, 3 dihydro-isoindol-2-yl)-piperidine-2, 6-dione (c)

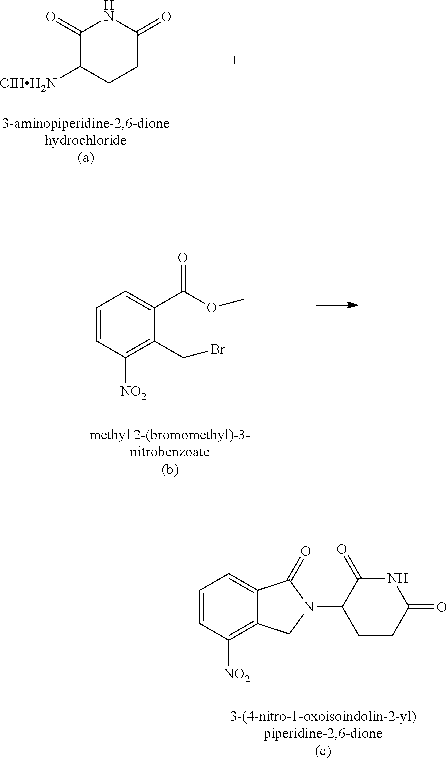

ii). reacting 3-(4-nitro-1-Oxo-1, 3 dihydro-isoindol-2-yl)-piperidine-2, 6-dione (c) with hydrogen gas at temperature ranging between 20-40° C. and normal atmospheric pressure in anhydrous medium in the presence of Palladium Carbon catalyst to get 3-(4-amino-1-oxoisoindolin-2-yl) piperidine-2,6-dione or Lenalidomide (I).

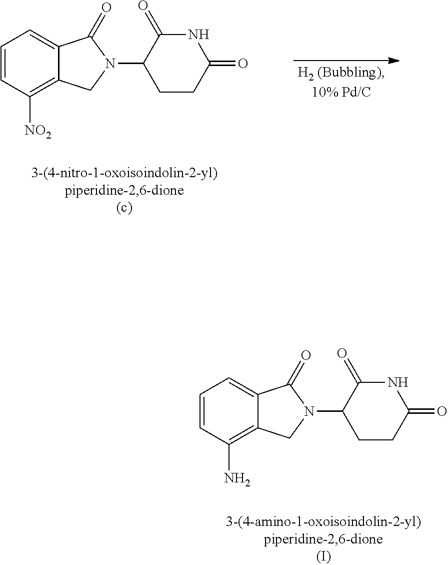 [SMILES: O=C1CCC(N2Cc3c(cccc3[N+](=O)[O-])C2=O)C(=O)N1 ; Nc1cccc2c1CN(C1CCC(=O)NC1=O)C2=O]

iii) optionally purifying compound (I) obtained in step ii) by combining with an alkylsulphonic acid in the presence of an aliphatic alcohol solvent at 25-35° C. under stirring for a time duration ranging between 30-60 minutes;
iv) filter and combine the filtrate with a mixture of an organic acid and an alkyl amine at temperature ranging between 25-35° C. for a time duration ranging between 20-50 minutes;
v) isolating the pure crystalline Form SRA.

Individual steps of the embodiments are detailed herein below.

In step i) process of combining the 3-aminopiperidine-2, 6-dione Hydrochloride (a) and methyl 2-(bromomethyl)-3-nitrobenzoate (b) is carried out at temperature ranging between 25-30° C. in the presence an organic amide solvent and triethyl amine to obtain 3-(4-nitro-1-Oxo-1, 3 dihydro-isoindol-2-yl)-piperidine-2, 6-dione (c).

The organic amide solvent used in step i) is selected from N, N-dialkyl (C₁-C₄) formamide.

The addition of the triethylmamine is preferably carried out slowly in the temperature range of 33-38° C. in order to ensure the completion of the reaction, improved yields and process efficiencies including high exothermicity.

In step ii), reaction is carried out of 3-(4-nitro-1-Oxo-1, 3 dihydro-isoindol-2-yl)-piperidine-2, 6-dione (c) with hydrogen gas at room temperature and normal atmospheric pressure in anhydrous medium in the presence of Palladium Carbon catalyst to get 3-(4-amino-1-oxoisoindolin-2-yl) piperidine-2,6-dione or Lenalidomide (I). This is one of the unique embodiment of the present invention compared from all prior arts reported processes performed this hydrogenation at elevated temperature as well as higher pressures (3-10 kg/cm2) in autoclave.

Inventors of the present application have surprisingly developed this safe and economically viable process of addition of hydrogen gas i.e. hydrogenation takes place via hydrogen gas bubbling at normal atmospheric pressure without involving any pressure creating device i.e. an autoclave.

Hydrogenation in the autoclave has disadvantage like limited batch size reaction leads to the formation of small amount of the resulting compound i.e. crude Lenalidomide.

In this step ii) reaction is performed in the presence of methane sulphonic acid which is a solubilising agent. Inventors of the present invention surprisingly noticed that the compound of formula 3-(4-nitro-1-Oxo-1, 3 dihydro-isoindol-2-yl)-piperidine-2, 6-dione must be completely dissolved in order to achieve the desired crystalline Form SRA of Lenalidomide.

Hydrogen gas bubbling has advantages like handling big batches besides improved yields/efficiencies and minimizing process related genotoxic impurities of plant operations due to incompletion of the reaction without compromising on the quality characteristics of final API (Active Pharmaceutical Ingredient) and improved process efficiencies and yields.

The polar organic solvent is selected from methanol, ethanol, acetone, isopropanol, methyl ethyl ketone, n-propanol, acetonitrile, DMSO and DMF.

Hydrogen gas bubbling in step ii) is advantageously carried out at temperature ranging between 30° C. to 35° C. and at normal atmospheric pressure while necessary to obtain the desired quality and yields. It was surprisingly observed by inventors that reaction carried out at this temperature and under normal atmospheric pressure results a new crystalline Form SRA of Lenalidomide.

In the subsequent step of the process of the present invention a treatment of the filtrate with activated charcoal and dimethylglyoxime is carried out to obtain the desired product.

In a particular embodiment addition of the solution A in 15 to 20 minutes at 25-30° C. Preparation of solution A is by the dissolution of citric acid and triethylamine in methanol. Citric acid is used to maintain the PH neutral.

Dimethylglyoxime and triethylamine both are used as a chelating agent to remove the metal impurities in order to get the higher yield of crystalline Form SRA of Lenalidomide.

Step iii) provides process for the preparation of pure crystalline Form SRA of Lenalidomide which involved the use of alkyl sulphonic acid in the presence of an aliphatic alcohol solvent at 25-35 C under stirring for a time duration ranging between 30-60 minutes.

In step iii), pure crystalline Form SRA of Lenalidomide formed—the washing of the cake (wet API) was conducted with pre cooled methanol.

The product obtained by the present invention is free of process related impurities, including unreacted intermediates, side products, degradation products and other medium dependent impurities.

In yet further embodiment the steps of combining citric acid and triethylamine, it comprises of slow addition of citric acid and triethylamine mixture, wherein citric acid and triethyl amine mixture prepared earlier by combining citric acid and triethylamine in methanol.

After combining this mixture, the solution may preferably be maintained at 10-15 C under stirring for a time duration of 3 hrs in order to get the higher yield of crystalline Form SRA of Lenalidomide.

The step of cooling the reaction mixture may be carried out for the mixture upto about 10-15° C. to attain the crystalline material precipitated out with no or minimal possible degradation for achieving the pure crystal form.

In another embodiment of the present invention, it provides a highly pure Crystalline Form SRA of Lenalidomide having purity exceeding of 99.9% (area % by HPLC).

The crystalline form obtained is a thermodynamically stable Form SRA exhibits an X-ray powder diffraction angle peaks expressed in 2θ° at approximately 7.7, 11.2, 14.2, 15.6, 19.9, 20.3, 23.9, 25.7, 28.2, 32.3, 33.4 2θ°±0.20 2θ° and having the melting point range between 265 to 270° C.

The process related impurities, including unreacted intermediates, side products, degradation products and other medium dependent impurities, that appears in the impurity profile of the Lenalidomide can be removed by the process of the present invention resulting in the formation highly pure Lenalidomide, which parallel leads to the formation of highly pure and stable crystalline Form SRA of Lenalidomide.

Anhydrous crystalline Form SRA of Lenalidomide obtained according to present invention shall be dried under vacuum to attain water content in the range between 0.1 to 0.5% w/w.

In yet further another embodiment isolation of the crystalline Form SRA of Lenalidomide may be carried out by filtration, solvent removal (extraction), layer separation, concentration, distillation, or a combination thereof, Process of the present invention avoids the formation of by-products and process related impurities in the formation of pure crystalline Form SRA of Lenalidomide.

Inventors of the present application performed six months stability for the novel crystalline Form SRA according to the following temperature and relative humidity conditions:

| S. No. | Temperature | Relative Humidity |
|---|---|---|
| A. | 25° C. ± 2° C. | 60% ± 5% |
| B. | 40° C. ± 2° C. | 75% ± 5% |

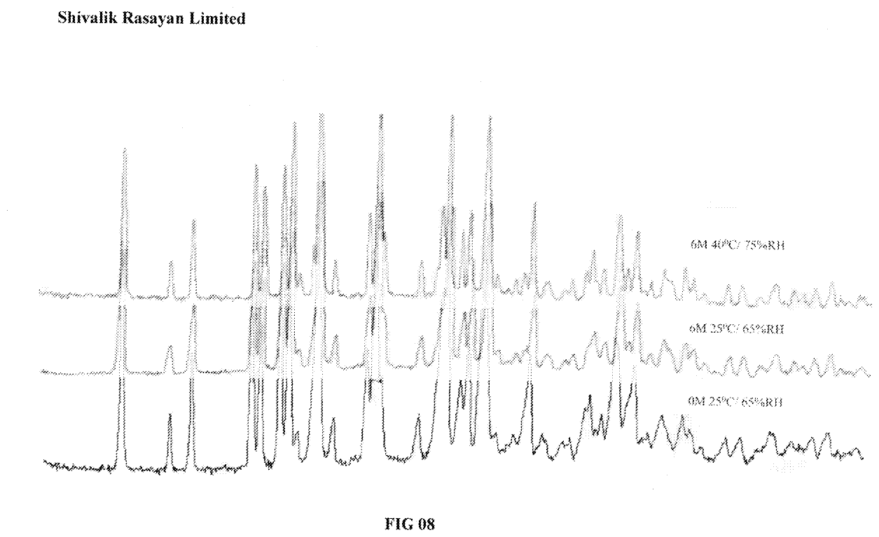

The below mentioned table shows the thermodynamic stability nature of the polymorphic Form SRA and the HPLC purity indicates to retain substantially pure form nature with purity exceeding 99.9% (% w/w by HPLC).

| Moisture Content (by | | | | | |
|---|---|---|---|---|---|
| KF method) | | | | | |
| Not more than 0.5% | | | | | |
| w/w | Initial | 1M | 2M | 3M | 6M |
| A | 0.23 | 0.22 | 0.22 | 0.23 | 0.23 |
| B | 0.23 | 0.21 | 0.22 | 0.22 | 0.22 |

| Assay (by HPLC; on | | | | | |
|---|---|---|---|---|---|
| anhydrous basis) | | | | | |
| Between 98.0 to | | | | | |
| 102.0% w/w | Initial | 1M | 2M | 3M | 6M |
| A | 100.38 | 99.50 | 99.41 | 99.55 | 99.47 |
| B | 100.38 | 99.77 | 99.64 | 100.02 | 99.47 |

| | After six months | After six months |
|---|---|---|
| | at 25° C. ± | at 40° C. ± |
| | 2/65 ± 5% | 2/75 ± 5% |
| XRPD Initial peaks: | RH condition | RH condition |
| 7.7, 11.2, 14.2, 15.6, | 7.7, 11.3, 14.3, 15.6, | 7.8, 11.3, 14.2, 15.6, |
| 19.9, 20.3, 23.9, 25.7, | 20.0, 20.3, 23.8, 25.7, | 20.0, 20.4, 23.8, 25.7, |
| 28.2, 32.3 and 33.4 ± | 28.3, 32.2 and 33.5 ± | 28.2, 32.3 and 33.5 ± |
| 0.2°2θ | 0.2°2θ | 0.2°2θ |

Wherein, A=25° C.±2/65±5% RH; B=40° C.±2/75±5% RH
   FIG. 8 illustrates the comparative X-ray powder diffraction (XRPD) pattern of Lenalidomide Form SRA of initial stage and after 6 month's stability (under 25° C.±2/65±5% RH and 40° C.±2/75±5% RH conditions), which reveals that the crystalline form remains unchanged and indicative of thermodynamic stable nature.

In yet further another embodiment, it provides that the crystalline Form SRA of Lenalidomide obtained by the processes of the present application may be formulated as solid compositions for oral administration in the form of capsules, tablets, pills, powders or granules useful in the treatment lung cancer and pancreatic cancer.

Crystalline Form SRA of Lenalidomide of the present invention may have one or more advantageous and desirable properties compared to the known Lenalidomide, which are not limited to better stability, solubility and quality parameter leading to improved storage and distribution.

In another embodiment, the pure crystalline Form SRA of Lenalidomide obtained by the processes of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Lenalidomide of the present application include, but are not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions derived from Crystalline Form SRA of Lenalidomide of the present application may also comprise to include the pharmaceutically acceptable carrier used for the preparation of solid dispersion, wherever utilized in the desired dosage form preparation.

While the present invention has been described in terms of its specific embodiments, certain modifications and equivalents will be apparent to those skilled in the art and are intended to be included within the scope of the present invention.

Certain specific aspects and embodiments of the present application will be explained in more detail with reference to the below examples, which are provided by way for illustration purpose only and should not be construed as limiting the scope of the invention in any manner.

### EXAMPLE

The following example illustrates the nature of the invention and is provided for illustrative purposes only and should not be construed to limit the scope of the present invention.

The process of preparation according to the present invention of crystalline Lenalidomide Form SRA may be demonstrated by examples as given below:

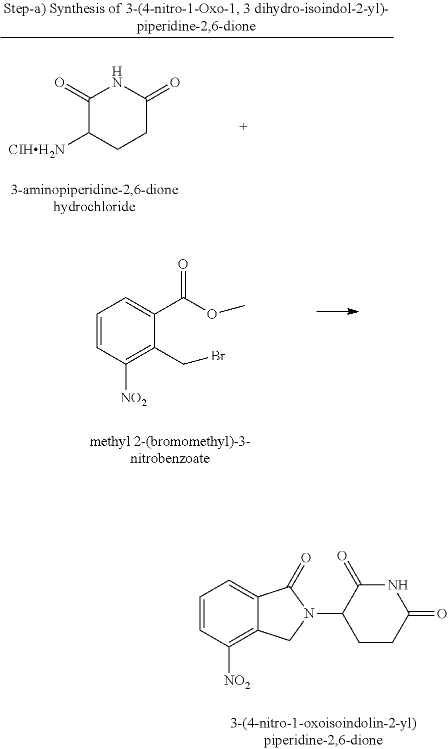

Charged 1500 ml N, N-dimethyl formamide in 5 litre four-necked round bottom flask at 25-30° C. under nitrogen atmosphere. Charged 100 gm 3-aminopiperidine-2, 6-dione Hydrochloride and 183.26 gm. of methyl 2-(bromomethyl)-3-nitrobenzoate into the round bottom flask at 25-30° C. under nitrogen atmosphere. Stirred the reaction mixture for 10 minutes at 25-30° C. under nitrogen atmosphere. Added 187 ml triethyl amine (TEA) slowly at 25-30° C. under nitrogen atmosphere. Added 1500 ml purified water slowly over a period of 15-20 minutes at 25-35° C. Maintained the reaction mass for 45 minutes at 25 to 35° C. Filtered the reaction mixture and washed the wet cake with 200 ml purified water. Suck dried the material under vacuum for 45 to 60 minutes. Charged wet cake again into round bottom flask and added 1000 ml purified water. Stirred the slurry mass for 20 to 30 minutes at 25 to 35° C. Filtered the solid obtained and washed the wet cake with 200 ml purified water. Suck dried the material under vacuum for 30 to 45 minutes. Charged wet cake again into round bottom flask and added 1000 ml methanol at 25-35° C. Stirred slurry mass for 25 to 30 minutes at 25 to 35° C. Filter the solid obtained and washed the wet cake with 2×100 ml methanol at 25-35° C. Suck dried wet cake for 45 to 60 minutes. at 25-35° C. Dried the material under vacuum at 60-65° C. for 12 hrs.

Yield (w/w): 1.5

HPLC purity: 98.0%

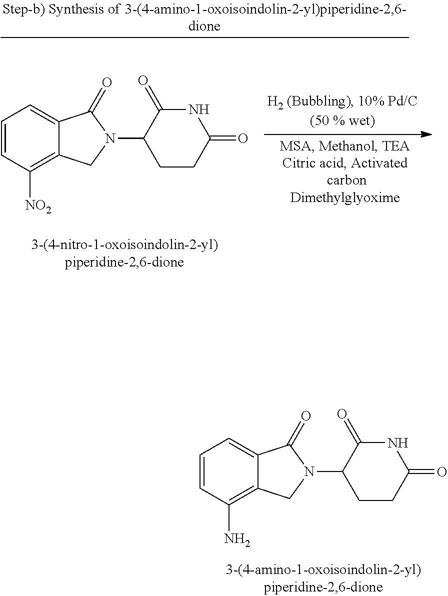 [SMILES: O=C1CCC(N2Cc3c(cccc3[N+](=O)[O-])C2=O)C(=O)N1 ; Nc1cccc2c1CN(C1CCC(=O)NC1=O)C2=O]

Charged 2500 ml methanol lot-2 to the flask at 25-35° C. under stirring. Charged 100 gm 3-(4-nitro-1-oxoisoindolin-2-yl) piperidine-2, 6-dione to a clean 5 litre four-necked round bottom flask at 25-35° C. under stirring. Bubbled nitrogen gas into the reaction mixture for 25 to 30 minutes at 25-35° C. Charged 50 gm methane sulfonic acid into the reaction mixture at 25-35° C. under stirring. Charged 10 gm 10% Palladium/Carbon (50% wet) into the reaction mixture at 25-35° C. Charged 500 ml of methanol for flushing under stirring under light nitrogen stream. Again bubbled the nitrogen gas into the reaction mixture for 25 to 30 minutes at 25-35° C. Raised the temperature of reaction mass to 30 to 35° C. Bubbled hydrogen gas into the reaction mass for 6 hrs at 30 to 35° C. without pressure. Prepared Hyflo bed using 25 gm hyflo and 200 ml methanol. After completion of the reaction filtered the reaction mass through hyflo bed and washed the bed with 100 ml methanol. Transferred the filtrate into another round bottom flask and charged 10 gm activated charcoal and 5 gm dimethylglyoxime into the filtrate at 25-35° C. Stirred the reaction mixture for 30 minutes at 25-35° C. Prepared Hyflo bed using 25 gm hyflo and 200 ml methanol. Filtered the reaction mass through hyflo bed and washed the bed with 100 ml of methanol. Charged the filtrated into another round bottom flask at 25-35° C. and charged the solution A in 15 to 20 minutes at 25-35° C.

### (Preparation of Solution A: Dissolve 50 gm of Citric Acid and 78.7 gm Triethyl Amine in 300 ml of Methanol at 25-35° C.)

After complete addition of solution A, cooled the reaction mixture to 10 to 15° C. under stirring. Stirred the precipitated solid for 3 hrs at 10 to 15° C. Filtered the solid and washed the wet cake with 200 ml of methanol (10-15° C.). Suck dried the wet cake for 25 to 30 minutes. Unloaded wet cake and charged into another round bottom flask at 25-30° C. Charged 400 ml of methanol into the above round bottom flask at 25-30° C. Stirred the slurry for 60 minutes. at 25-30° C. Filtered the solid and washed the cake with 200 ml methanol 25-30° C. Suck dried the wet cake for 45 to 60 minutes. Unloaded the wet cake and dried the material at 60 to 65° C. under vacuum for about 12 hrs. Unloaded the material.

Yield (w/w): 0.65

HPLC purity: 98.0%

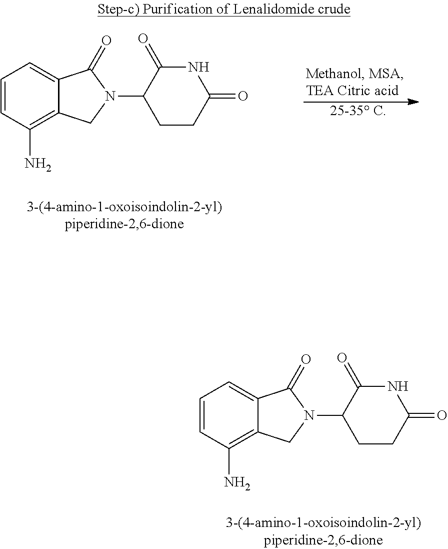 [SMILES: Nc1cccc2c1CN(C1CCC(=O)NC1=O)C2=O ; Nc1cccc2c1CN(C1CCC(=O)NC1=O)C2=O]

Charged 3000 ml methanol and 100 gm crude Lenalidomide to a clean 5 litre four-necked round bottom flask at 25-35° C. under stirring. Charged 37.1 gm methane sulfonic acid at 25-35° C. under stirring. Stirred the reaction mass for 30 minutes at 25-35° C. Filtered the reaction mass through filter paper at 25-35° C. Charged the filtrate into another multi-neck round bottom flask at 25-35° C. Charged the solution A in the above round bottom flask at 25-35° C. in 20-25 minutes under stirring. Suck dried the wet cake for 30 to 45 minutes. Charged 1000 ml methanol into separate round bottom flask at 25-35° C. Charged the wet cake again into the multi-neck round bottom flask at 25-35° C. Stirred the slurry for 30 to 45 minutes at 25-35° C. Filtered the solid and washed the cake with 200 ml of methanol. Suck dried the wet cake for 30 to 45 minutes. Unloaded wet cake and dried the material under vacuum at 60-65° C. for 12 hrs.

Yield (w/w): 0.80

HPLC purity: 99.9%.

The above mentioned example, which is provided by way of illustration, should not be construed as limiting the scope of the invention with respect to parameter/s, ingredient/s and quantities use etc.

We claim:

1. A process for preparing 3-(4-amino-1-oxoisoindolin-2-yl) piperidine-2,6-dione or Lenalidomide (I) crystalline Form SRA 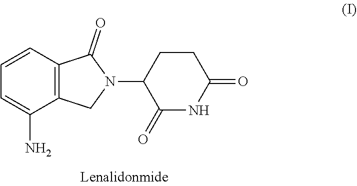 [SMILES: Nc1cccc2c1CN(C1CCC(=O)NC1=O)C2=O]
characterized by X-ray powder diffraction angle peaks at 7.7, 11.2, 14.2, 15.6, 19.9, 20.3, 23.9, 25.7, 28.2, 32.3, 33.4 2θ°±0.20 2θ° and DSC endothermic peak ranging between 265 to 270° C.;
Comprising the steps of:
i) combining the compound of formula (a) with compound of formula (b) in an organic amide solvent and triethyl amine to get 3-(4-nitro-1-Oxo-1, 3 dihydro-isoindol-2-yl)-piperidine-2,6-dione (c); 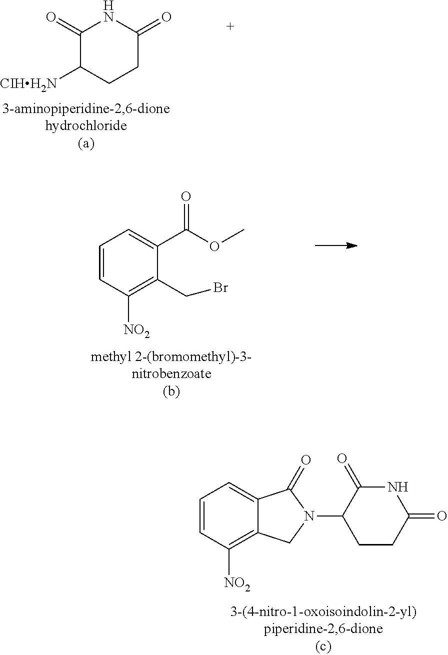
ii). reacting 3-(4-nitro-1-Oxo-1, 3 dihydro-isoindol-2-yl)-piperidine-2, 6-dione (c) with hydrogen gas at temperature ranging between 20-40° C. and normal atmospheric pressure in anhydrous medium in the presence of Palladium Carbon catalyst to get 3-(4-amino-1-oxoisoindolin-2-yl) piperidine-2,6-dione or Lenalidomide (I); 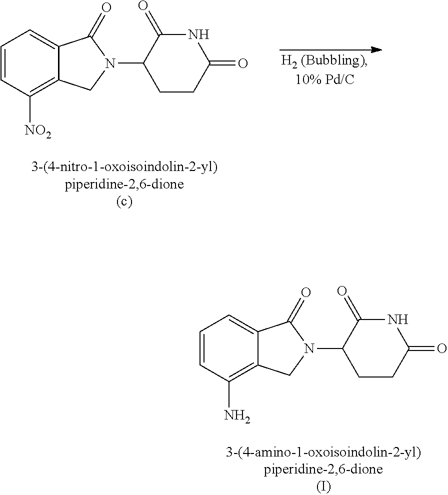 [SMILES: O=C1CCC(N2Cc3c(cccc3[N+](=O)[O-])C2=O)C(=O)N1 ; Nc1cccc2c1CN(C1CCC(=O)NC1=O)C2=O]
iii). optionally purifying compound (I) obtained in step ii) by combining with an alkylsulphonic acid in the presence of an aliphatic alcohol solvent at 25-35° C. under stirring for a time duration ranging between 30-60 minutes;
iv). filter and combine the filtrate with a mixture of an organic acid and an alkyl amine at temperature ranging between 25-35° C. for a time duration ranging between 20-50 minutes;
v). isolating the pure crystalline Form SRA.

2. A process for preparing 3-(4-amino-1-oxoisoindolin-2-yl) piperidine-2, 6-dione or Lenalidomide (I) crystalline Form SRA as claimed in claim 1, wherein an organic amide solvent used in step i) is selected from N,N-dialkyl (C₁-C₄) amide.

3. A process for preparing 3-(4-amino-1-oxoisoindolin-2-yl) piperidine-2, 6-dione or Lenalidomide (I) crystalline Form SRA as claimed in claim 1, wherein hydrogenation in step ii) takes place via hydrogen gas bubbling at normal atmospheric pressure.

4. A process for preparing 3-(4-amino-1-oxoisoindolin-2-yl) piperidine-2, 6-dione or Lenalidomide (I) crystalline Form SRA as claimed in claim 1, wherein organic acid used in step iv) is selected from citric acid, tartaric acid, lactic acid or malic acid.

5. A process for preparing 3-(4-amino-1-oxoisoindolin-2-yl) piperidine-2, 6-dione or Lenalidomide (I) crystalline Form SRA as claimed in claim 1, wherein alkylamine used in step iv) is selected from triethylamine, diisopropyl amine or diisopropyl ethyl amine.

6. 3-(4-amino-1-oxoisoindolin-2-yl) piperidine-2,6-dione or Lenalidomide (I) as anhydrous crystalline Form SRA characterized by having water content of 0.1-0.5% w/w; X-ray powder diffraction angle peaks at 7.7, 11.2, 14.2, 15.6, 19.9, 20.3, 23.9, 25.7, 28.2, 32.3, 33.4 2θ°±0.20 2θ°, an IR absorption spectral peaks at 3410 cm⁻¹, 3347 cm⁻¹, 2962 cm⁻¹, 2912 cm⁻¹, 1642 cm⁻¹, 1607, 1452 cm⁻¹, 1411 cm⁻¹, 1209 cm⁻¹, 1163 cm⁻¹, 1014 cm⁻¹ and DSC endothermic peak ranging between 265 to 270° C.

7. 3-(4-amino-1-oxoisoindolin-2-yl) piperidine-2, 6-dione or Lenalidomide (I) crystalline Form SRA as claimed in claim 6, characterized by X-ray powder diffraction pattern according to FIG. 1, TGA Thermo gravimetric analysis according to FIG. 3, IR absorption spectrum according to FIG. 4, DSC isothermal pattern according to FIG. 5 and High performance Liquid chromatographic purity according to FIG. 6.

8. 3-(4-amino-1-oxoisoindolin-2-yl) piperidine-2, 6-dione or Lenalidomide (I) crystalline Form SRA as claimed in claim 6 having purity of greater than 99.9% (area % by HPLC).
